(19) United States
(12) Patent Application Publication
(10) Pub. No.: US 2020/0163788 A1
(43) Pub. Date: May 28, 2020

(54) Hamstring Assist Device

(71) Applicant: Doak Matthew Ostergard, Lincoln, NE (US)

(72) Inventor: Doak Matthew Ostergard, Lincoln, NE (US)

(21) Appl. No.: 16/693,643

(22) Filed: Nov. 25, 2019

Related U.S. Application Data
(60) Provisional application No. 62/771,191, filed on Nov. 26, 2018.

(51) Int. Cl. A61F 5/01; A61F 13/08; A61L 15/12; A61F 5/30

(52) U.S. Cl. CPC A61F 5/0106; A61F 5/0109; A61F 5/30; A61L 15/12; A61F 13/08

(57) ABSTRACT

The present invention relates to the field of sports, performance, and orthopedic braces, and more specifically, sports, performance, and orthopedic braces made for the hamstring. This Hamstring-Assist Device aids in restoring function to the affected muscle during recovery from an injury by mimicking the structural characteristics of the associated muscles.

### BACKGROUND

The hamstring consists of three large muscles which run along the back of the upper leg extending from the pelvis to the top of the bones of the lower leg. Together the biceps femoris, semitendinosus, and semimembranosus function to achieve hip extension and knee flexion, ultimately propelling the body forward in order to run, walk, or jump.

Most hamstring injuries are typically strains or tears to the associated tendons or muscles. These injuries frequently occur in athletes, especially in those who participate in sports that require sudden explosive movements such as sprinting, jumping, or lunging. However, hamstring injuries can also occur more gradually during slower movements that stretch the hamstring beyond its capacity. These injuries can vary in severity; however, all result in a loss of muscle function, causing pain, swelling, and weakness that can persist for weeks, resulting in a strength deficit and, most often, decreased performance for the athlete.

Many hamstring injuries respond well to non-surgical treatments. Typical non-surgical treatments include rest, ice, compression, elevation, immobilization, and physical therapy. Additional developments in non-surgical hamstring treatments are needed to provide compensation for inadequate muscle function, lowered muscle activation, and increased range of motion while allowing the athlete the opportunity to continue performing when experiencing a strength deficit.

### BRIEF DRAWING DESCRIPTIONS

FIG. 1 is an orthogonal anterior view of the Hamstring-Assist Device.

FIG. 2 is an orthogonal side view of the Hamstring-Assist Device.

FIG. 3 is an orthogonal posterior view of the Hamstring-Assist Device depicting the femoral wrap.

FIG. 4 is a perspective side view of the Hamstring-Assist Device in a position of hip extension and knee flexion.

FIG. 5 is an exploded view of the Hamstring-Assist Device depicting the individual components.

FIG. 6 is an additional orthogonal posterior view of the Hamstring-Assist Device worn in conjunction with compression shorts.

### BRIEF DESCRIPTION

The Hamstring-Assist Device, which protects and aids the hamstring in hip extension and knee flexion by mimicking the structural characteristics of the associated muscles, is disclosed.

The present invention includes a circumferential waist belt, a fastener system, a medial leg strap, a lateral leg strap, and an anti-migration strap. In another embodiment, the Hamstring-Assist Device can be worn in combination with compression shorts to provide compression to the affected leg. In another embodiment, the Hamstring-Assist Device includes a femoral wrap to provide compression to the affected leg. The femoral wrap is secured around the upper leg of the wearer to provide warmth and compression to the affected leg.

The circumferential waist belt is cinched around the waist to a preferred fit and is secured to the body by a fastening system. In one embodiment, the circumferential waist belt fastener system is a double D-ring. In other embodiments, the fastener system could include hook-and-loop or a single D-ring, a clip, a belt-hole mechanism, or a tensioning system. The fastener system allows the wearer to adjust the circumferential waist belt circumference and prevents any loosening or tightening of the circumferential waist belt during use. A semi-rigid, pressure-dispersion pad is adhered to the circumferential waist belt to alleviate D-ring pressure from the waist of the wearer. In another embodiment, the pressure-dispersion pad could include a padded sleeve.

Adjustable medial and lateral leg straps attach to the circumferential waist belt and extend down the leg to the ankle. In one embodiment, the leg straps are made of neoprene. In another embodiment, the leg straps are made of a two- or four-way stretch material.

While in a prone position, with the hip extended and the knee slightly flexed, the medial leg strap is aligned with the medial portion of the leg, near the buttocks, and the lateral leg strap is aligned with the lateral side of the leg, two to three inches toward the lateral side of the affected leg. The medial leg strap is pulled toward the medial side of the shin of the affected leg, crossing the hamstring, creating tension. The medial leg strap is pulled across the anterior of the shin, just below the knee cap. The medial leg strap continues to spiral down the lower leg around the ankle and is secured to itself through hook-and-loop fasteners.

The lateral leg strap is pulled toward the lateral side of the shin of the affected leg, crossing the medial leg strap at the hamstring, creating tension. The lateral leg strap is pulled across the anterior of the shin just below the knee cap. The lateral leg strap continues to spiral down the leg, and is around the ankle, over the medial leg strap through hook-and-loop fasteners. This crisscross pattern provides a natural spring-back effect to the leg, assisting the hamstring by mimicking natural muscle function.

To prevent migration, the medial leg strap and lateral leg strap are secured below the kneecap of the wearer with an anti-migration strap. In one embodiment, the anti-migration strap is made of neoprene and secured to the leg of the wearer through hook-and-loop fasteners.

Indications for the Hamstring-Assist Device include hamstring sprains, strains, pulls, tweaks, pain; weakness in hip extension and knee flexion; among others.

### DETAILED DESCRIPTION

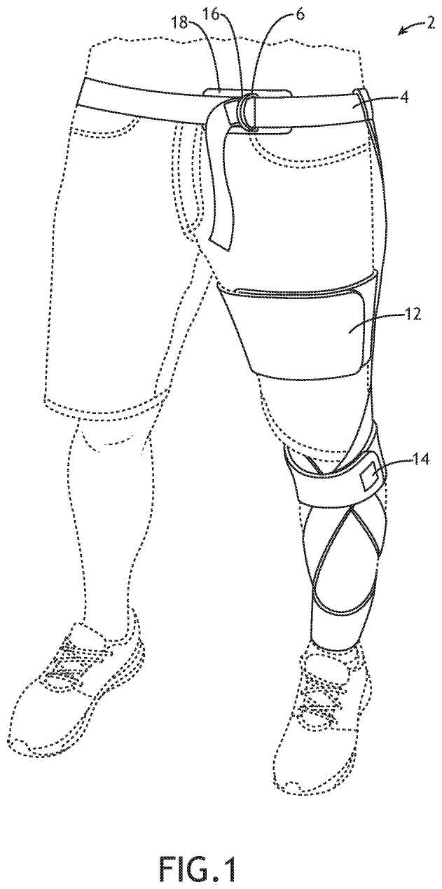

The Hamstring-Assist Device 2, shown in FIG. 1, protects and aids the hamstring in hip extension and knee flexion by mimicking the structural characteristics of the associated muscles, is disclosed.

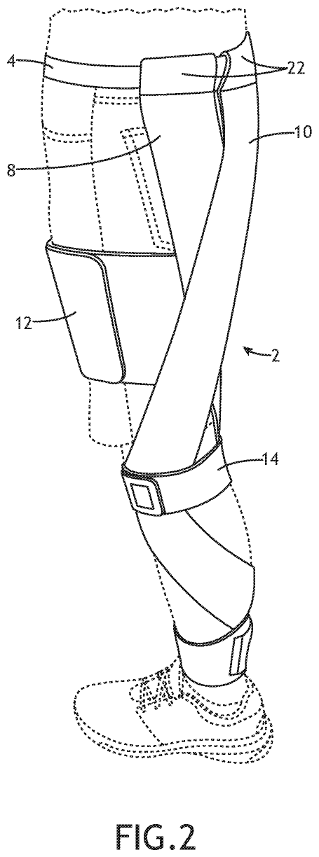

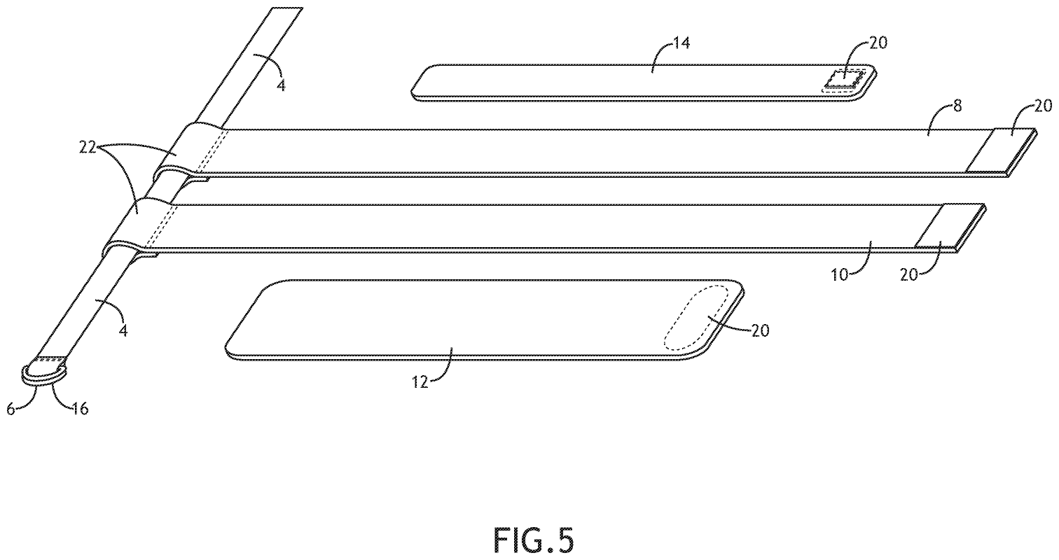

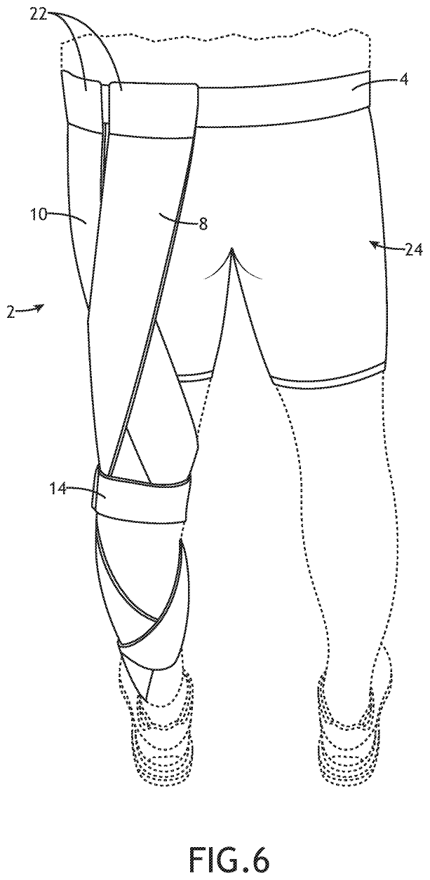

The present invention includes a circumferential waist belt 4, fastener system 6, a medial leg strap 8, lateral leg strap 10, and anti-migration strap 14, shown in FIG. 5. In another embodiment, the Hamstring-Assist Device 2 can be worn in combination with compression shorts 24 to provide compression to the affected leg, shown in FIG. 6. In another embodiment, the Hamstring-Assist Device 2 includes a femoral wrap 12, shown in FIG. 2, to provide compression to the affected leg. The femoral strap 12 is secured around the upper leg of the wearer to provide warmth and compression and worn underneath the medial leg strap 8 and lateral leg strap 10. In one embodiment, the femoral strap 12 is secured by hook-and-loop fasteners 20, as shown in FIG. 5.

The circumferential waist belt 4 is cinched around the waist of the wearer to a preferred fit and secured to the body by a fastener system 6. In one embodiment, the fastener system 6 is a double D-ring 16, shown in FIG. 1. In this embodiment, upon wrapping the circumferential waist belt 4 around the body, the circumferential waist belt 4 is fed through the double D-ring 16 and adheres to itself through hook-and-loop fasteners. In another embodiment, the fastener system 6 could include a buckle or a tensioning system. The double D-ring 16 allows the wearer the ability to adjust the circumferential waist belt 4 circumference and prevents any loosening or tightening of the circumferential waist belt 4 during use. In one embodiment, the double D-ring 16 is made of a metal material with a likeness to nickel. In another embodiment, the circumferential waist belt 4 is made of a polyester-like fiber or nylon. A semi-rigid, pressure-dispersion pad 18 is adhered to the circumferential waist belt to alleviate D-ring 16 from the waist of the wearer, as shown in FIG. 1. In another embodiment, the pressure dispersion pad could include a padded sleeve.

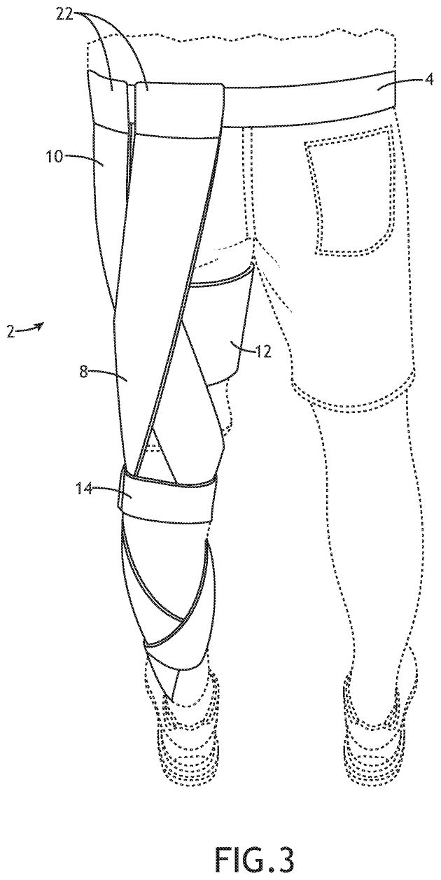

The medial leg strap 8 and the lateral leg strap 10, in one embodiment, are made of neoprene and fastened to the circumferential waist belt 4 by looping around the circumferential waist belt 4 and are stitched to themselves, shown in FIG. 3. The loops 22, as shown in FIG. 3, allow the medial leg strap 8 and lateral leg strap 10 to move and be repositioned along the circumferential waist belt 4.

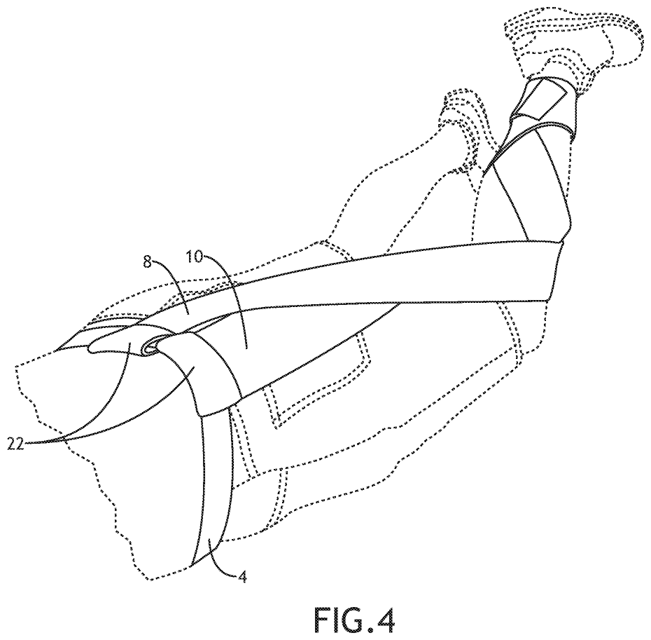

While in a prone position, as shown in FIG. 4, with the hip extended and the knee slightly flexed, the medial leg strap 8 and lateral leg strap 10 are aligned with the medial portion of the leg, near the buttocks, and lateral side of the leg, two to three inches toward the lateral side of the affected leg. The medial strap 8 is then pulled toward the medial side of the shin of the affected leg, crossing the hamstring, creating tension. The medial leg strap 8 is then pulled across the anterior of the shin, just below the knee cap. The medial leg strap 8 continues to spiral down the lower leg around the ankle and is secured to itself through hook-and-loop fasteners 20.

The lateral leg strap 10 is pulled toward the lateral side of the shin of the affected leg, crossing the medial leg strap 8 at the hamstring, creating tension. The lateral leg strap 10 is then pulled across the anterior of the shin just below the knee cap. The lateral leg strap 10 continues to spiral down the leg, and is secured around the ankle, over the medial leg strap 8 through hook-and-loop fasteners 20, as shown in FIG. 4. This crisscross pattern provides a natural spring-back effect to the leg, assisting the hamstring by mimicking natural muscle function.

To prevent migration, the medial leg strap 8 and lateral leg strap 10 are secured below the kneecap of the wearer with an anti-migration strap 14, wrapped around the lower leg over the medial leg strap 8 and lateral leg strap 10, as shown in FIG. 3. The anti-migration strap 14 acts as an anchoring point to prevent the Hamstring-Assist Device 2 from migrating during use. In one embodiment, the anti-migration strap 14 is made of neoprene and secured to the leg of the wearer through hook-and-loop fasteners 20.

Indications for the Hamstring-Assist Device 2 include hamstring sprains, strains, pulls, tweaks, pain; weakness in hip extension and knee flexion; among others.

1: I claim a device for supporting a hamstring, comprising:
a circumferential waist belt;
a fastener system;
leg straps attached to the circumferential waist belt; and
an anti-migration strap.

2: I claim a device for supporting a hamstring, comprising:
a circumferential waist belt,
a fastener system,
a medial leg strap,
a lateral leg strap,
a femoral wrap, and
an anti-migration strap.

3: I claim a device of claim 2 wherein the circumferential waist belt is made of a polyester-like fiber or nylon.

4: I claim a device of claim 2 wherein the fastener system includes a double D-ring, single D-ring, a clip, a belt-hole mechanism, or a tensioning system.

5: I claim a device of claim 2 wherein the leg straps are made of neoprene.

6: I claim a device of claim 2 wherein the anti-migration strap is secured to the leg of the wearer through hook-and-loop fasteners.

7: I claim a device of claim 6 wherein the anti-migration strap is made of neoprene.

8: I claim a device for supporting a hamstring, comprising:
a circumferential waist belt,
a fastener system,
a medial leg strap,
a lateral leg strap,
compression shorts, and
an anti-migration strap.

8: I claim a device for supporting a hamstring, comprising:
a circumferential waist belt,
a pressure-dispersion pad,
a fastener system,
a medial leg strap,
a lateral leg strap,
compression shorts, and
an anti-migration strap.

9: I claim a device of claim 8 wherein the pressure dispersion pad is a padded sleeve.

10: I claim a device for supporting a hamstring, comprising:
a circumferential waist belt,
a pressure-dispersion pad,
a fastener system,
a medial leg strap,
a lateral leg strap,
a femoral wrap, and
an anti-migration strap.

12: I claim a device for supporting a hamstring, comprising:
a circumferential waist belt secured to the body through a double d-ring,
a semi-rigid, pressure-dispersion pad adhered to the circumferential waist belt,
a fastener system,
a medial leg strap is adjustably adhered to the circumferential waist belt,
a lateral leg strap is adjustably adhered to the circumferential waist belt,
a femoral wrap secured to the leg of the wearer through hook-and-loop fasteners, and
an anti-migration strap secured to the leg of the wearer through hook-and-loop fasteners.

13: I claim a method for supporting a hamstring, compromising:
placing a circumferential waist belt configured around the waist of a wearer,
cinching said circumferential waist belt to a preferred fit,
securing said circumferential waist belt configured to the waist of a wearer through a fastening system,
aligning a medial leg strap and lateral leg strap, fastened to said circumferential waist belt, with the medial side of a leg and lateral side of a leg,
pulling said medial leg strap toward the medial side of a shin, crossing a hamstring, continuing to pull said medial leg strap crossing the anterior of a shin, just below a kneecap,
spiraling down a lower leg around an ankle,
securing said medial strap to itself through hook-and-loop fasteners,
pulling said lateral leg strap toward the lateral side of a shin, crossing said medial leg strap at a hamstring,
pulling said lateral leg strap across the anterior of a shin below a kneecap,
spiraling down a leg around an ankle,
securing said lateral strap around an ankle, over said medial strap, and
securing said medial leg strap and said lateral leg strap below a kneecap of a wearer with an anti-migration strap, wrapped around a lower leg over said medial leg strap and said lateral leg strap.

14: I claim a method of claim 13 wherein the method to support a hamstring can be worn in combination with compression shorts.

15: I claim a method of claim 13 wherein the method to support a hamstring can be worn in combination with a femoral strap.
